(19) United States
(12) Patent Application Publication
(10) Pub. No.: US 2025/0318921 A1
(43) Pub. Date: Oct. 16, 2025

(54) INTRAOCULAR LENS (IOL) DELIVERY SYSTEM

(71) Applicant: Adaptilens, Inc., Chestnut Hill, MA (US)

(72) Inventors: Liane CLAMEN, Chestnut Hill, MA (US); Trevor RUGGIERO, Chestnut Hill, MA (US); Brett THOMES, Chestnut Hill, MA (US); Dan STANLEY, Chestnut Hill, MA (US)

(73) Assignee: Adaptilens, Inc., Chestnut Hill, MA (US)

(21) Appl. No.: 19/177,190

(22) Filed: Apr. 11, 2025

Related U.S. Application Data
(60) Provisional application No. 63/632,839, filed on Apr. 11, 2024.

(51) Int. Cl. A61F 2/16

(52) U.S. Cl. CPC A61F 2/167

(57) ABSTRACT

An intraocular lens (IOL) delivery system allows for a larger IOL, such as an Accommodating Intraocular Lens and/or a fluid-filled IOL, to be delivered into the capsular bag of an eye through a small incision in the eye in a controlled manner without damaging the eye, the lens, or the incision site. The delivery systems include a syringe-like mechanism that directly or indirectly moves the IOL through a cartridge. Various mechanisms of inserting the IOL are described, including guiding the IOL into the eye's capsular bag, or conveying the IOL into the eye's capsular bag.

### CROSS REFERENCE TO RELATED APPLICATION

This application claims the benefit of priority to the following applications, the entire contents of all of which are incorporated herein by this reference for all purposes:
U.S. Provisional Application Ser. No. 63/632,839, entitled “INTRAOCULAR LENS (IOL) DELIVERY SYSTEM” filed on Apr. 11, 2024.

### FIELD OF THE INVENTION

The disclosure generally relates to intraocular lenses and, more particularly, to systems for delivering intraocular lenses into the eye.

### BACKGROUND OF THE INVENTION

The lens capsule, or the capsular bag, of the eye is a thin membrane around the eye's natural lens that holds the lens in a central position within the eye and helps give the lens its shape. The capsular bag comprises an anterior and posterior capsule. Attached to the periphery of the capsule are tiny string-like structures called zonules which attach to the ciliary muscle. The ciliary muscle plays a critical role in accommodation, the process that allows the eye to adjust focus for near, intermediate, and distance vision. For near vision, the ciliary muscle contracts and reduces the tension on the zonules. This allows the lens to become rounder, thus increasing its refractive power so the eye can focus on near objects. For distance vision, the ciliary muscle relaxes, increasing tension on the zonules and pulling the capsular bag taut. Thus, the lens flattens and has decreased refractive power, enabling the eye to focus on distant objects.

As people reach about age 45 and older, the lens becomes stiffer and less able to change shape and power. With this age-related condition, called presbyopia, people must use glasses or contact lenses to see clearly at near and intermediate. At about the age of 75, the natural lens becomes stiffer and cloudy, a condition known as a cataract. The treatment for this is cataract surgery, during which the cataract is removed and an artificial intraocular lens or IOL is inserted into the lens capsule in place of the cloudy cataractous lens.

The purpose of an accommodating intraocular lens (AIOL) is to restore the ability of the human eye to accommodate under typical visual stimuli. An accommodating intraocular lens works with the ciliary muscles to allow people to see over a range of distances. The AIOL is surgically inserted into the capsular bag of the eye during cataract surgery, after the cataractous lens is removed. An AIOL may be made of material(s) that imitate the optical and mechanical properties of a healthy, young lens.

An AIOL may comprise a lens body in the form of a hollow shell which is expanded with a filler material. Examples of this type of AIOL are disclosed in Applicant's prior U.S. Pat. Nos. 10,278,810 and 11,678,976 which both describe fluid filled, accommodating IOLs comprising a capsular shell or interface enclosing an optically acceptable medium. The medium provides shape to the capsular interface, optical power, and a physiologic response to the suspensory ligament. An example of a filling material to be used in this lens is described in WO2022246198A1

More recently an “all-in-one” AIOL design comprising a soft, flexible material that does not require in situ filling is being developed. International Publication WO2023225332A1 describes polymers that can be used to make an all-in-one AIOL that does not require a shell. International Publication WO2024233709A2 also describes an optically clear bottlebrush copolymer that can be used to make an all-in-one AIOL that does not require a shell.

Typically, an intraocular lens (IOL) is loaded into an injection molded polymeric cartridge, and force is directly applied to the IOL, e.g., by a plunger, such that the IOL is pushed through the cartridge directly into the eye of a patient. Many IOL are semi-rigid, with various haptic mechanism extending from the lens body perimeter, and respond to force differently than accommodating IOL which are soft liquid filled and readily deform or compress under traditional delivery forces from syringe-like injectors. Because of the different structural and behavioral characteristics of liquid filled AIOLs, previously available IOL delivery systems are often not optimally suited for delivering AIOLs into the eye of a patient.

Accordingly a need exists for an accommodating intraocular lens delivery system that optimizes the behavior characteristics of an AIOL lens.

Accordingly a further need exists for an accommodating intraocular lens delivery system capable of safely delivering a soft and flexible AIOL directly into the capsular bag of the patient's eye.

A further need exists for an accommodating intraocular lens delivery system that allows for easy and safe deployment.

Another need exists for an accommodating intraocular lens delivery system that does not require a larger incision in the eye.

### SUMMARY OF THE INVENTION

An intraocular lens (IOL) delivery system allows for a larger IOL, such as an Accommodating Intraocular Lens and/or a fluid-filled IOL, to be delivered into the capsular bag of an eye through a small incision in the eye in a controlled manner without damaging the eye, the lens, or the incision site. The delivery system includes a syringe-like mechanism that directly or indirectly moves the IOL through a cartridge. Various mechanisms of inserting the AIOL are described, including guiding the AOL into the eye's capsular bag, or conveying the AIOL into the eye's capsular bag.

In accordance with disclosure, an inserter cartridge for delivery of an accommodating intraocular (AIOL) lens into the eye of a patient includes an inserter cartridge body, a plunger, a thin film, and a metallic wire. The thin film surrounds and compresses the soft, flexible AIOL. The plunger is configured to extrude the intraocular lens from the inserter cartridge body. The intraocular lens is packaged in the thin film and the thin film has a metallic wire or plastic filament embedded into a portion thereof. The plunger assists in inserting the film-wrapped AIOL directly into the eye's capsular bag. Once the AIOL is above the capsular bag, voltage is applied to the metallic wire or plastic filament causing melting or severing from the thin film and enabling the intraocular lens to be delivered from the inserter cartridge body into the capsular bag. In such embodiment, the inserter cartridge includes electrical contacts positioned on the inserter cartridge body to provide electrical connection between the metallic wire or plastic filament and an external power source.

In embodiments, the plunger may include a soft tip which pushes the intraocular lens through the inserter cartridge tip and delivers it into the eye's capsular bag. A volume on the interior of the inserter cartridge body between the soft tip and the thin film surrounding the AIOL may be filled with a non-compressible fluid. The plunger transfers pressure to the non-compressible fluid, thereby causing the intraocular lens to enter the eye.

In accordance with one embodiment of the invention, a method of delivering an intraocular lens into an eye of a subject includes packaging an intraocular lens in a thin film, loading the intraocular lens packaged in the thin film into an inserter cartridge body, applying force to an external surface of a plunger, and applying a voltage to a metallic wire to sever the thin film to permit the intraocular lens to be delivered from the inserter cartridge body directly into the capsular bag of the eye. The plunger is configured to apply pressure to a volume on the interior of the inserter cartridge body between a soft tip and a thin film surrounding the intraocular lens, and the volume is filled with a non-compressible fluid. The plunger applies pressure to the thin film surrounding the intraocular lens filled with a non-compressible fluid.

In accordance with one embodiment, a method of delivering an intraocular lens into an eye includes an inserter cartridge body with a stiff, narrow segment that has a diameter<3.5 mm. The narrow segment has a thin film, that is compressed or twisted to a small diameter, attached to its distal tip. This thin film portion of the inserter expands to a larger diameter as the IOL enters the eye, allowing the surgeon to guide the IOL into the capsular bag and also allowing for the least compression of the IOL possible

In accordance with disclosure, a method of delivering an intraocular lens into an eye utilizes a compartmentalization process. An AIOL is segmented using a thin thread or thin film placed proximate one end of the lens. The thread constricts the shell body into two sections through which the internal liquid can still flow. A compressive device is applied to the remaining length of the lens, forcing all of the liquid into one portion of the segmented shell interior. The thread is then used to create a tighter constriction, ensuring that the filler liquid stays within one segmented portion of the shell interior. The other portion of the shell interior, i.e. the remaining length of the shell is compressed into a small diameter tube with a short section of the AIOL extending distally and exposed at the tip of the tube. The exposed portion of the AIOL is then inserted directly into the eye's capsular bag. Once the tip of the AIOL is positioned in the capsular bag, the constricting thread is released, thereby allowing the proximal end of the AOL to contract while expanding the distal end thereof and self-extruding the remainder of the AIOL into the eye's capsular bag.

In accordance with another aspect of the disclosure, a system for delivery of an intraocular lens comprises, in combination: an injector body having a lumen opening at a distal end thereof, the lumen having a minimum internal diameter; and an accommodating intraocular lens comprising a flexible shell body filled with a filler liquid, the shell body is configured to at least partially assume an uncompressed maximum diameter substantially larger than the minimum internal diameter of the injector body lumen and further configured to at least partially assume a compressed diameter less than the minimum internal diameter of the injector body lumen. In embodiments, the injector body further comprises a mechanism for moving the accommodating intraocular lens toward the distal end of the injector body lumen. In embodiments, the accommodating intraocular lens is disposed within the injector body lumen distally of the mechanism for moving and proximally of the distal end of the injector body lumen. In embodiments, the mechanism for moving the accommodating intraocular lens toward the distal end of the injector body lumen comprises a plunger slidably disposed within the injector body lumen. In embodiments, the mechanism for moving the accommodating intraocular lens toward the distal end of the injector body lumen further comprises a liquid disposed within the injector body lumen between the plunger and the accommodating intraocular lens. In embodiments, the injector body lumen comprises a wide diameter proximal section and a narrower diameter distal section in fluid communication therebetween, and wherein the plunger transfers distally directed force thereon through the fluid and to the accommodating intraocular lens causing compression of the shell body at a point of transition from the wide diameter proximal section to the narrower diameter distal section. In embodiments, the mechanism for moving the accommodating intraocular lens toward the distal end of the injector body lumen further comprises a plurality of film strips disposed intermediate the minimum internal diameter of the injector body lumen and the at least partially compressed diameter the shell body. In embodiments, the mechanism for moving the accommodating intraocular lens toward the distal end of the injector body lumen further comprises a mechanism for applying distally directed forces on the plurality of film strips. In embodiments, the plurality of film strips transfer distally directed force thereon to the accommodating intraocular lens.

In accordance with another aspect of the disclosure, an intraocular lens delivery system comprises: an injector body having a lumen opening at a distal end thereof, and an electrical contact exposed on the injector body lumen and configured to electrically couple a power source associated with the delivery system with an intraocular lens at least partially compressed by a wrapper material and an electrically conductible element. In embodiments, the intraocular lens delivery system is in combination an accommodating intraocular lens comprising a flexible shell body filled with a filler liquid, the shell body at least partially compressed by a wrapper material and an electrically conductible element connectable to a power source.

In accordance with another aspect of the disclosure, system for delivery of an intraocular lens comprises, in combination: an injector body having a lumen opening at a distal end thereof; and an accommodating intraocular lens comprising a flexible shell body filled with a filler liquid, the shell body at least partially compressed by a wrapper material and an electrically conductible element connectable to a power source. In embodiments, the intraocular lens delivery system is in combination with a power source associated with the delivery system. In embodiments, at least a portion of wrapper material comprises a film. In embodiments, the electrically conductible element comprises a wire. In embodiments, coupling the electrically conductible element to the power source causes the wrapper to at least partially melt. In embodiments, the electrically conductible element is electrically coupled to the power source outside injector body lumen. In embodiments, a plunger is slidably disposed within the injector body lumen to transfer distally directed force thereon to the accommodating intraocular lens.

In accordance with another aspect of the disclosure, system for delivery of an intraocular lens comprises: an injector body having a lumen opening exteriorly at a distal end thereof, the lumen defined by an interior wall; and a flexible tubular restraint coupled proximate the distal end for directing expansion of an intraocular lens emerging from the lumen in a controlled manner. In embodiments, the flexible tubular restraint is attached to the interior wall of the lumen. In embodiments, a flexible tubular restraint is attached to at an exterior portion of the injector body. In embodiments, flexible tubular restraint is configured to prevent symmetric radial expansion of the intraocular lens. In embodiments, flexible tubular restraint is twisted into a predetermined expansion pattern. In embodiments, flexible tubular restraint is any of wrapped, twisted or perforated in a predetermined pattern to bias expansion of the intraocular lens in a downward direction relative to an axis of the lumen upon the intraocular lens emerging from the distal end. In embodiments, flexible tubular restraint comprises a foldable film. In embodiments, an accommodating intraocular lens comprising a flexible shell body filled with a liquid.

In accordance with another aspect of the disclosure, a method for delivering an AIOL, the AIOL comprising a flexible shell body filled with an incompressible liquid, the method comprises: A) configuring a flexible tubular restraint attached to a distal end of a semi-rigid lumen into a predetermined expansion pattern, and B) advancing an AIOL in a compressed state out of a distal end of a semi-rigid lumen and into a flexible tubular restraint at a rate to prevent symmetric radial expansion of the intraocular lens upon emerging from the lumen. In embodiments, B) comprises: B1) preventing expansion of the intraocular lens in an upward direction relative to an axis of the lumen upon the intraocular lens emerging from the distal end.

In accordance with another aspect of the disclosure, a method for delivering an intraocular lens comprises: A) loading an intraocular lens into a lumen of an injector body, the lumen having an open distal end, and B) advancing the intraocular lens distally through the lumen towards the open distal end without applying force to a proximal facing surface of the intraocular lens as positioned in the lumen.

In accordance with another aspect of the disclosure, a system for delivery of an intraocular lens comprises: an injector body having a lumen opening exteriorly at a distal end thereof, the lumen defined by an interior wall surface; a plurality of strips disposed adjacent the interior wall surface; and a mechanism attached to the injector body for advancing the plurality of strips distally out of the distal end. In embodiments, each of the plurality of strips has a first side facing the lumen interior wall surface and a second oppositely facing side. In embodiments, the first side of each of the plurality of strips has a different co-efficient of friction than the second side of the respective strip relative to the lumen interior wall surface. The first side of each of the plurality of strips has a lower co-efficient of friction than the second side of the respective strip relative to the lumen interior wall surface. In embodiments, each of the plurality of strips comprises a loop which co-acts with the mechanism for advancing the loop distally relative to the lumen interior wall surface. In embodiments, the intraocular lens delivery system is in combination with an accommodating intraocular lens comprising a flexible shell body filled with a filler liquid, the accommodating intraocular lens positionable within the lumen but separated from portions of the interior wall surface by the plurality of strips.

In accordance with another aspect of the disclosure, a method for delivering an AIOL comprising a flexible shell body filled with an incompressible liquid, comprises first constricting the shell body so that a majority of the incompressible liquid in its interior is disposed in a first portion of the shell body and a remaining portion of the incompressible liquid is disposed in a second portion of the shell body but still in fluid communication with the first portion of the shell body. Next, the practitioner advances the second portion of the shell body through an incision having a diameter greater than the second portion of the shell body but less than that of the first portion of the shell body and at least partially beyond the opening. Finally, the constriction is removed unconstricting the shell body while the first portion of the shell body is exterior of the opening so that the incompressible liquid disposed in the first portion of the shell body flows into the second portion of the shell body, thereby advancing the balance of the AIOL into the capsular bag of the eye.

### BRIEF DESCRIPTION OF THE DRAWINGS

Those skilled in the art should more fully appreciate advantages of various embodiments of the invention from the following “Detailed Description” discussed with reference to the drawings summarized immediately below.

FIG. 1A illustrates conceptually a top view of an accommodating intraocular lens according to embodiments described herein.

FIG. 1B illustrates conceptually a side view of the accommodating intraocular lens of FIG. 1A.

FIG. 1C illustrates conceptually a cross-sectional view of the accommodating intraocular lens of FIG. 1B as viewed along line A-A.

FIG. 1D illustrates schematically a delivery system with OVD fluid according to embodiments of the disclosure.

FIG. 2A illustrates a cross-sectional schematic of a lens wrapped in a film thread to assist in compression and delivery of IOL according to embodiments of the disclosure.

FIG. 2B illustrates a cross-sectional schematic of film being used to compress the IOL, and then the film being cut back at incision.

FIG. 2C illustrates a cross-sectional schematic of films used to equally distribute stretch in shell during compression according to embodiments of the disclosure.

FIG. 2D illustrates a cross-sectional schematic of films used to equally distribute stretch in shell during compression, and the IOL exiting the device according to embodiments of the disclosure.

FIG. 3A is a photograph of a prototype delivery system according to embodiments of the disclosure.

FIG. 3B is a photograph of a prototype delivery system according to embodiments of the disclosure.

FIG. 3C is a photograph of a prototype delivery system according to embodiments of the disclosure.

FIG. 3D is a photograph of a prototype delivery system according to embodiments of the disclosure.

FIG. 4A illustrates a cross-sectional schematic of how an IOL is segmented using a thin thread or film placed 2 mm from one end of the lens according to embodiments of the disclosure.

FIG. 4B illustrates a cross-sectional schematic how the thread is then used to make a constricted opening through which the internal fluid can still flow according to embodiments of the disclosure.

FIG. 4C illustrates schematically how a compressive device is applied to the remaining length of the lens, forcing all of the fluid into the segmented portion of the shell according to embodiments of the disclosure.

FIG. 4D illustrates schematically how the thread is then used to seal the constriction, ensuring that the fluid stays within the segmented portion of the shell according to embodiments of the disclosure.

FIG. 4E illustrates schematically how a 2 mm section of the shell is exposed at the tip of the tube and this is then inserted into the eye according to embodiments of the disclosure.

FIG. 4F illustrates schematically how once the tip of the shell is positioned in the capsular bag, the sealing thread is released and the fluid can flow back into the compressed portion of the lens according to embodiments of the disclosure.

FIG. 5A illustrates a cross-sectional schematic how the compressive device is a thin film surrounding the lens with two free ends running between a set of spring-loaded jaws.

FIG. 5B illustrates schematically how pulling on the free ends of the film in an alternating fashion simultaneously rolls and compresses the lens allowing the stretch to be evenly distributed through the shell.

FIG. 5C illustrates schematically a device for packaging the IOL in the thin film according to embodiments of the disclosure.

FIG. 6A illustrates a cross-sectional schematic of an inserter cartridge according to embodiments of the disclosure.

FIG. 6B illustrates schematically an IOL wrapped in film being inserted into the eye through a limbal incision and into the eye's capsular bag according to embodiments of the disclosure.

FIG. 6C illustrates schematically the thin film being cut when voltage is applied to the wire near the thin film according to embodiments of the disclosure.

FIG. 6D illustrates schematically the plunger pushing the rest of the lens into the capsular bag according to embodiments of the disclosure.

FIG. 6E is a photograph of a device for packaging the lens in a thin film according to embodiments of the disclosure.

FIG. 7 is a perspective view of a “football” shaped cartridge tip according to embodiments of the disclosure.

FIG. 8A illustrates a cross-sectional schematic of a delivery system for reducing the length and increasing the diameter of a cartridge tip according to embodiments of the disclosure.

FIG. 8B illustrates a cross-sectional schematic of a delivery system for reducing the length and increasing the diameter of a cartridge tip according to embodiments of the disclosure.

### DETAILED DESCRIPTION

An intraocular lens (IOL) delivery system allows for a larger IOL, such as an Accommodating Intraocular Lens and/or a fluid-filled IOL, to be delivered into the capsular bag of an eye through a small incision in the eye in a controlled manner without damaging the eye, the lens, or the incision site. The delivery system includes a syringe-like mechanism that directly or indirectly moves the IOL through a cartridge. Various mechanisms of inserting the AIOL are described, including guiding the AIOL into the eye's capsular bag, or conveying the AIOL into the eye's capsular bag.

Various embodiments of a delivery system disclosed herein may be utilized with an accommodating intraocular lens (AIOL) the restores the ability of the human eye to accommodate under typical visual stimuli by materials and geometries that mimic the mechanical and optical properties of the young Human Crystalline Lens (HCL). AIOLs are intended to change shape and thus change power to provide a continuous range of vision. In embodiments, AIOLs have a lens body comprising a shell with a liquid filler, intended for placement in the capsular bag of the eye. As used herein, the disclosed AIOL lens bodies are capable of accommodation in response to ciliary muscle stimulus without further haptic structures coupled exteriorly of the perimeter 25 of the lens body 15, e.g. springs, wings, pontoons, auxiliary reservoirs, or other structures, etc. used to maintain the lens body within the capsular bag.

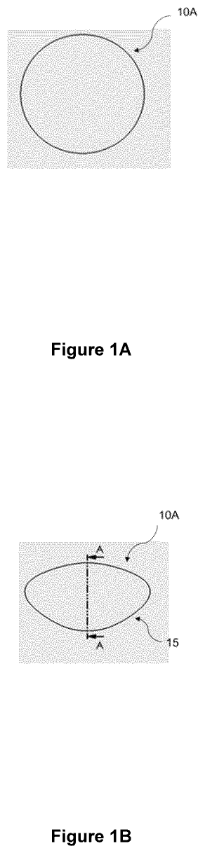

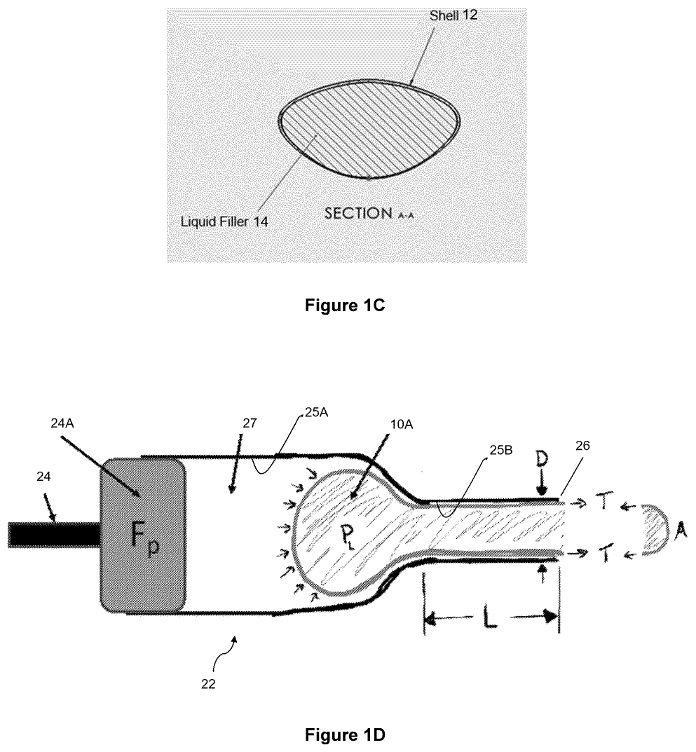

As shown in FIGS. 1A-C, an AIOL 10A has a lens body 15 comprising a shell 12 and an incompressible filler liquid 14. The materials of the shell 12 and filler 14 may be selected to closely match the HCL to provide maximal shape and thus power change. The overall dimensions of the AIOL 10A are selected to match the average dimensions of an HCL. In embodiments, the filler 14 may comprise a proprietary bottle-brush polymer (BBP), such as those disclosed in any of US20240287232A1 or International Publication WO2022246198A1 or International Publication WO2024233709A2. The shell 12 may comprise a silicone rubber or acrylate or similar material that provides safety, stability,

FIG. 1D illustrates schematically the delivery system 20 comprising a cylindrical injector body 22 having a lumen 25A extending therethrough to a distal end 26, similar to a syringe-like mechanism. A plunger 24 is slidably disposed in lumen 25 and directly or indirectly pushes the AIOL 10A through the lumen 25. The injector body 22 applies forces directly or indirectly to the lens depending on the design and properties of the IOL. The forces applied by the plunger 24 advance the lens through the lumen 25 and directly into the capsular bag of the eye.

As illustrated, the tip of plunger 24A, e.g., solid or soft-tip, that fills a lumen 25 and engages with the AIOL 10A, allowing the application of force directly to the AIOL 10A for advancement through the lumen 25. As the AIOL advances, the AIOL is folded or compressed to allow for small incision delivery (<3.5 mm). The disclosed system 20 can deliver IOLs through small incisions (<2.2 mm) and large fluid-filled IOLs through small incisions (<3.5 mm). In embodiments he lumen 25A has a larger interior diameter portion and a smaller interior diameter portion 25B, with compression of the AIOL 10A occurring proximate the transition in diameter between portion 25A and 25B. In embodiments, a portion of the lumen 25 may comprise an injector cartridge insertable into cylindrical injector body 22 in a manner which maintains fluid communication throughout the length of the lumen 25.

In embodiments, an AIOL is loaded into a cylindrical injector body 22 or injection cartridge and advanced manually with a surgical instrument, such as an Ophthalmic Viscosurgical Device (OVD). An OVD 27 fluid is applied behind the lens. The soft-tip plunger 24 fills the lumen 25 and transfers applied force onto the OVD 27 fluid which, in turn, transfers the forces (Fp) uniformly to the proximally facing surface of the AIOL 10A. Because the AIOL is soft and flexible, the lens does not move when pushed. Rather, the lens deforms, building tension in the body of shell 12 of the AIOL 10A and increasing the internal pressure. The internal pressure on the filler liquid 14 in the lens pushes on the area of the lens at the distal end 26. The resulting tension advances the lens forward if such tension is larger than the frictional forces within the interior diameter of lumen 25. The frictional forces are a function of the internal pressure of the lens and the area of the lumen 25. The required friction coefficient for motion is linearly correlated with diameter and inversely correlated with length. For example, for a lumen 25 having a 2.25 mm interior diameter and 6 mm length, the friction coefficient should be below 0.094. Examples of friction coefficient, μ, for various materials are set forth below:

$$Polyethylene on Polyethylene : μ = 0.2$$

$$PTFE on PTFE : μ = 0.04 - 0.1$$

The cylindrical injector body 22 is intended to fold or compress the shell 12 of the AIOL, thereby: 1) allowing the lens to be folded or compressed for delivery through a small incision (e.g. 2.5-3.5 mm); 2) allowing for improved control of the implantation process, including: a.) delivery with low forces required to push plunger by surgeon; b.) delivery with well controlled, consistent speed by surgeon; c.) delivery directly into the capsular bag; and d.) designed such that the lens does not rotate during delivery; 3) delivering the AIOL without damage to the IOL; 4) delivering the AIOL without negatively impacting the incision site or wound; and 5) delivering the AIOL without damaging any of the structures inside the eye such as the capsular bag and the cornea.

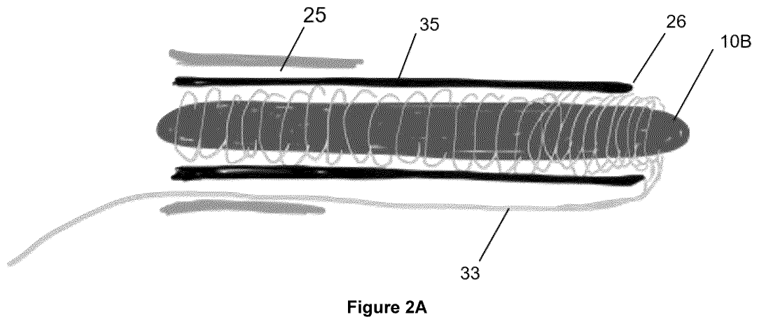

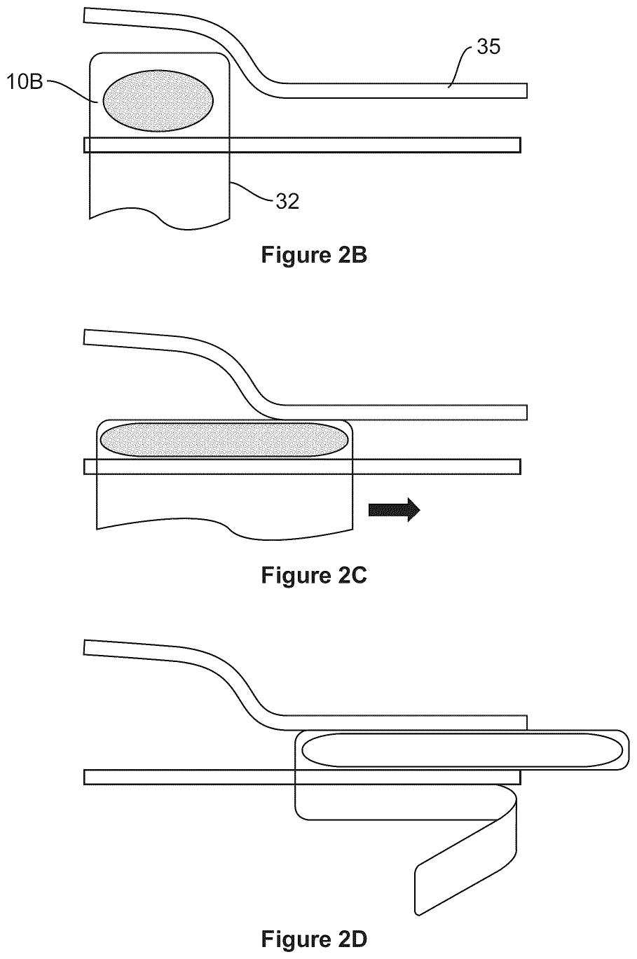

According to another aspect of the disclosure, referring to FIGS. 2A-C, an IOL 10B is subjected to an initial step to compress or prepare the lens for delivery into the eye. In embodiments IOL 10B may be either a traditional IOL or an AIOL as described herein. The IOL is wrapped using a thread or fiber 33 which produces a compressed, elongated IOL. This configuration or assembly is then placed into the cartridge or onto the cartridge 35. Upon advancement of the plunger, the fiber 33 is pulled out the tip of the cartridge 35 and pulls the lens through the cartridge by unravelling the fiber. As the fiber 33 is unraveled, the lens expands out the tip of the cartridge and is delivered into the eye of the subject. As the fiber exits the distal tip, the fiber is pulled back into a portion of the device to not impact any tissue or structures within the eye or incision.

In embodiments, a thin film 32 wraps around the lens within a cartridge 35. The free ends of the film 32 go through slits (not shown) in the cartridge 35 such that by pulling on the film, the lens is compressed. By sliding the free ends of the film forward, the lens is advanced through the cartridge and into the capsular bag. The free ends of the film are cut by the cartridge at the incision site, allowing them to peel back and only the top of the film covering the lens is advanced into the eye. After insertion of the lens, the cartridge is removed along with the cut film that went into the eye. FIG. 2A schematically shows a lens wrapped in a film thread to assist in compression and delivery of IOL. FIG. 2B schematically shows a film being used to compress the IOL, and then the film being cut back at incision, and then the IOL exiting the device. FIG. 2C schematically shows films used to equally distribute stretch in the IOL shell during compression according to embodiments of the disclosure.

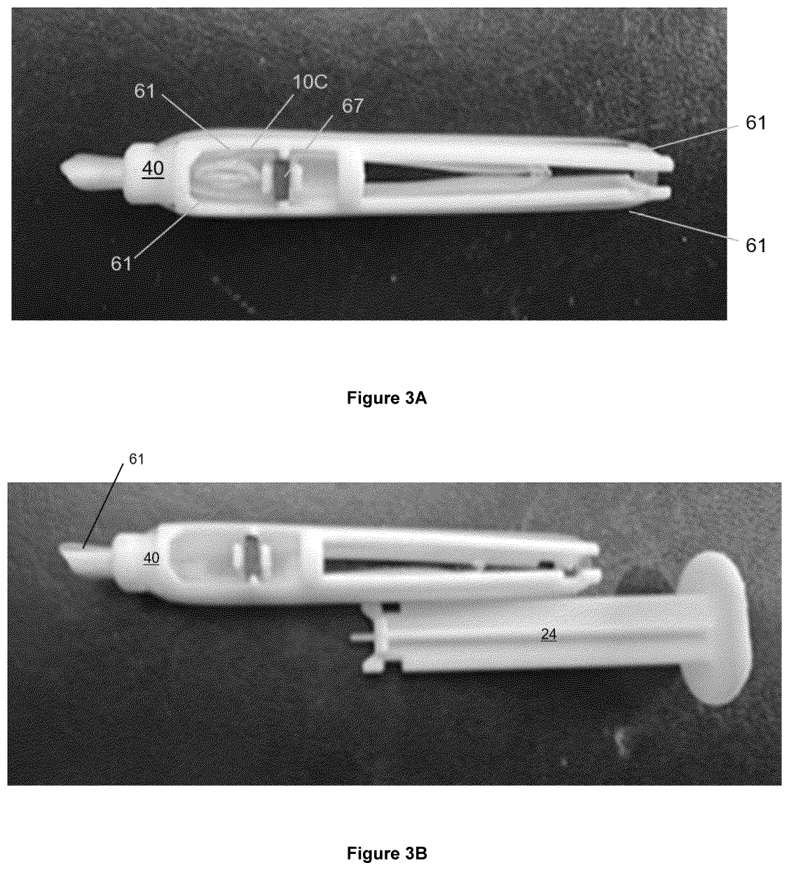

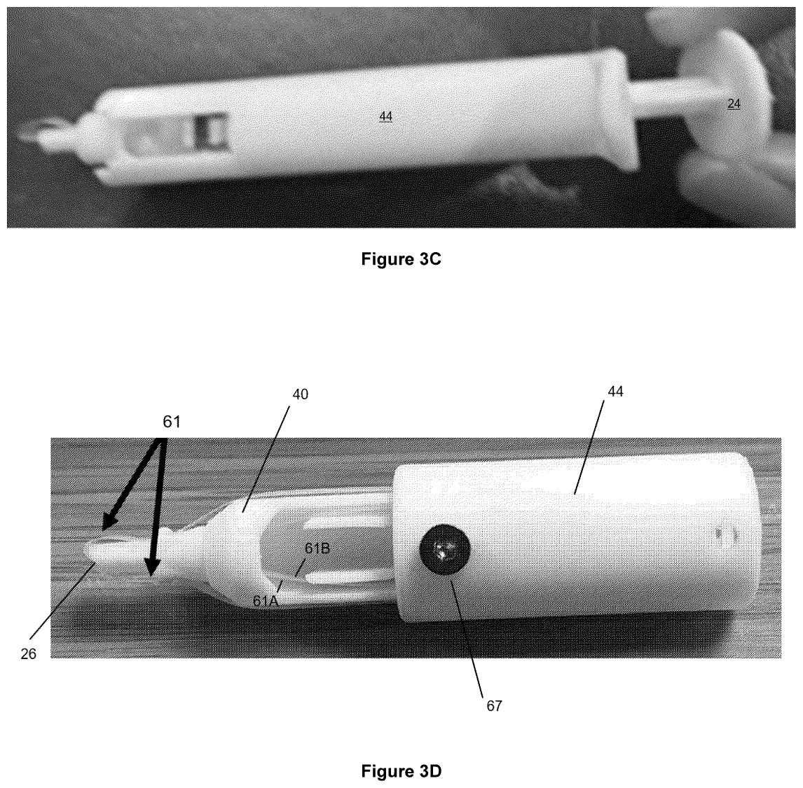

According to another aspect of the disclosure, referring to FIGS. 3A-C, an IOL 10C is delivered via a system without applying force to a proximal facing surface of the intraocular lens as positioned in the lumen. With this system, an intraocular lens 10C loaded into a lumen of an injector body 20 and advanced distally through the lumen 25 towards the open distal end 26 without applying force to a proximal facing surface of the intraocular lens as positioned in the lumen 25.

The system for delivery of the intraocular lens 10C comprises an injector body 20 having a lumen 25 opening exteriorly at a distal end 26 thereof, the lumen defined by an interior wall surface. The system further comprises a plurality of strips 61 disposed adjacent the lumen interior wall surface 25A and a mechanism 66 attached to the injector body 20 for advancing the plurality of strips 61 distally out of the distal end 26. Each of the plurality of strips has a first side 61A facing the lumen interior wall surface 25A and a second oppositely facing side 61B. The first side 61A of each of the plurality of strips has a lower co-efficient of friction than the second side 61B of the respective strip relative to the lumen interior wall surface 25A. In embodiments, each of the plurality of strips 61 comprises a loop which co-acts with the mechanism for advancing 66 the loop distally relative to the lumen interior wall surface. In embodiments, the mechanism for advancing 66 may comprise a plunger having either a forward or retrograde motion, a thumbwheel, or other device capable of drawing the strips distally out of the distal end 26 of the injector body 20 without the necessity of direct force or pressure being applied to a proximal facing surface of the intraocular lens as positioned in the lumen. Instead, the lower co-efficient of friction of first side 61A of strips 61 against interior wall surface 25A and the higher co-efficient of second side 61B of each strip 61 against the IOL shell results in any distally directed forces from outside the distal end 26 pulling distally outward on the strips 61 to cause the IOL to be compressed within lumen 25 and advance toward distal end 25 and emerge therefrom without the need for direct force or pressure being applied from behind the intraocular lens on the proximal facing surface, as positioned in the lumen 25. In embodiments, the intraocular lens delivery system is combined with an accommodating intraocular lens 10C positionable within the lumen but separated from of the lumen interior wall surface 25A by the plurality of strips 61.

Referring to FIG. 3A-C, the IOL 10C is loaded into a delivery system comprising an injection cartridge 40, with a plunger 24, and film strips 61 formed into continuous respective loops. The cartridge retains film strips 61, optionally coated with a hydrophilic coating on the side 61A facing the interior surface 25A of the lumen 25, that can be used to pull the IOL 10C through the cartridge 40. Injection cartridge 40 includes two projections extending proximally to engage the plunger 24. A divider 67 is disposed in lumen 25 injection cartridge 40 to maintain separation of the film strips 61 prior to their respective contact with IOL 10C. Note that the strip 61 do not contact IOL 10C along the surface of the shell thereof that is facing in the proximal direction away from distal end 26. In this embodiment, forward motion of the plunger 24 within the lumen 25 is translated to pulling of the film strips 61 out of the lumen 25 along with the IOL 10C that may be disposed in the lumen 25. FIG. 3D is a photograph of a cartridge 40 including a handpiece 44 that has the mechanism 66 for advancing strips 61 implemented with a thumbwheel turning mechanism. When the thumbwheel mechanism 66 is turned, the film strips 61 are advanced distally out of distal end 26 and the IOL 10C is pulled forward and out of the distal end 26. The IOL 10C is pulled and compressed by the film 61 to allow for small incision delivery, e.g. <3.5 mm. In embodiments, as the film strips 61 exit the tip of the cartridge 40, the strips 61 are pulled back into the lens delivery device. A wound guard may be employed to protect the incision site from the film strips 61.

According to another aspect of the disclosure, a method of delivering an intraocular lens into an eye utilizes a compartmentalization process. An AIOL 10D is segmented using a thin thread or film 45 placed proximate one end of the lens. The thread constricts the body of shell 12 into two sections through which the internal liquid can still flow. A compressive device is applied to the remaining length of the lens, forcing all of the liquid into one portion of the segmented shell interior. The thread 45 is then used to create a tighter constriction, ensuring that the filler liquid 14 stays within one segmented portion 46 of the shell interior. The other portion of the shell interior 48, i.e. the remaining length of the shell 12, is compressed into a small diameter tube with a short section of the AIOL 10D extending distally and exposed at the distal end 26 of the tube. The exposed portion 48 of the AIOL 10D is then inserted directly into the eye's capsular bag. Once the tip of the AIOL is positioned in the capsular bag, the constricting thread 45 is released, thereby allowing the proximal portion, portion 46, of the AOL 10D to contract while expanding the distal portion, portion 48, thereof and self-extruding the remainder of the AIOL 10D into the eye's capsular bag.

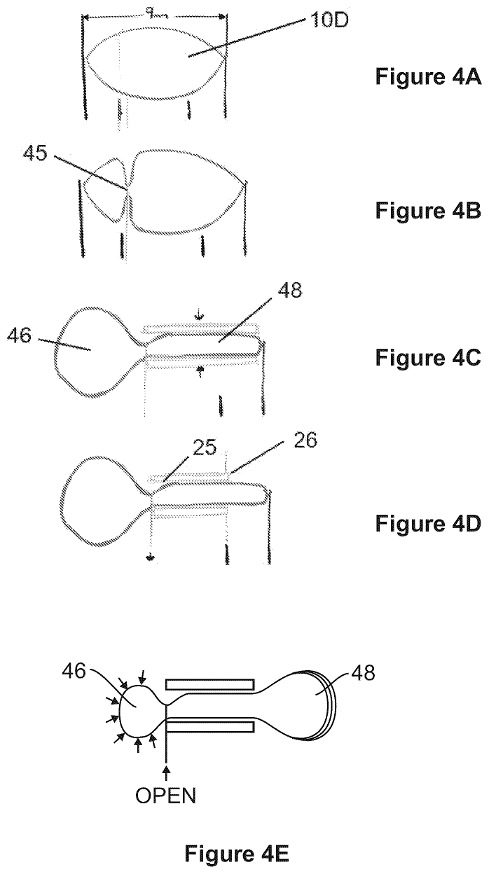

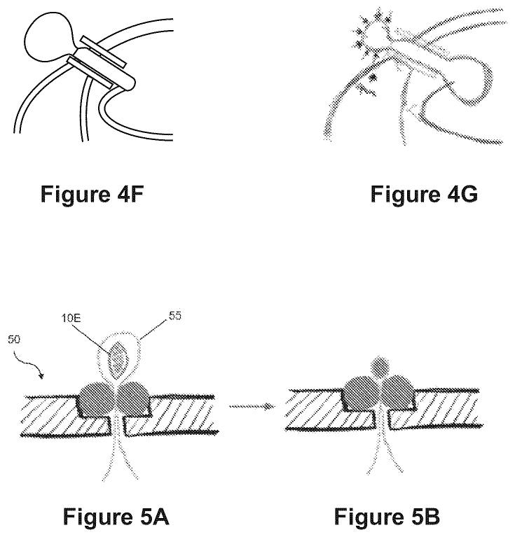

In embodiments, an AIOL 10D, similar to AIOL 10A, may be inserted into the eye using the compartmentalization process, as illustrated with reference to FIGS. 4A-4F. In FIG. 4A, the AIOL is segmented into first and second fluidly communicating interior sections using a constriction mechanism, such as thin thread, placed proximate, e.g. 2 mm from one end of the AIOL perimeter. This position may be determined by calculating the minimum surface area of AIOL shell that can withstand the full fluid volume of the lens without exceeding the elongation limit for the shell material, plus a factor of safety. In an exemplary embodiment, an AIOL 10D, based on the volume calculations, may have a small portion of the shell 12 able to accommodate most of the fluid volume inside 10D. If the lens is segmented into compartments, then the shell should never break. In this example, 2 mm of lens length holds the majority of the interior volume, allowing 5 mm of transition length.

In FIG. 4B, the constriction mechanism or thread 45 is used to make a constricted opening between first and second fluidly communicating interior sections. In FIG. 4C, a separate, compressive mechanism is applied to the remaining length of the lens body (second interior section), forcing most of the liquid filler into the segmented, first interior section of the shell. In FIG. 4D, the constriction mechanism or thread 45 is then used to seal the constriction, ensuring that the filler stays within the first interior section of the shell. The remaining length of the shell is then substantially empty and can be compressed into a small diameter tube such as lumen 25, either injection molded or formed from a thin film. In FIG. 4E, a short, e.g. 2 mm, section of the AIOL 10D, is exposed at the distal end 26 of the tube and inserted directly into the eye's capsular bag. In FIG. 4F, once the end of the AIOL is positioned in the capsular bag, the constriction mechanism or thread is released, thereby allowing filler liquid from the proximal end of the AIOL to flow and expand into the distal end of the AIOL and self-extrude into the eye's capsular bag. Because 2 mm of the shell may be left exposed at the tip and because this corresponds to the shell area that can withstand the full volume of the internal lens fluid, the proximal end of the lens can be compressed without concern of overstretching or bursting the distal end.

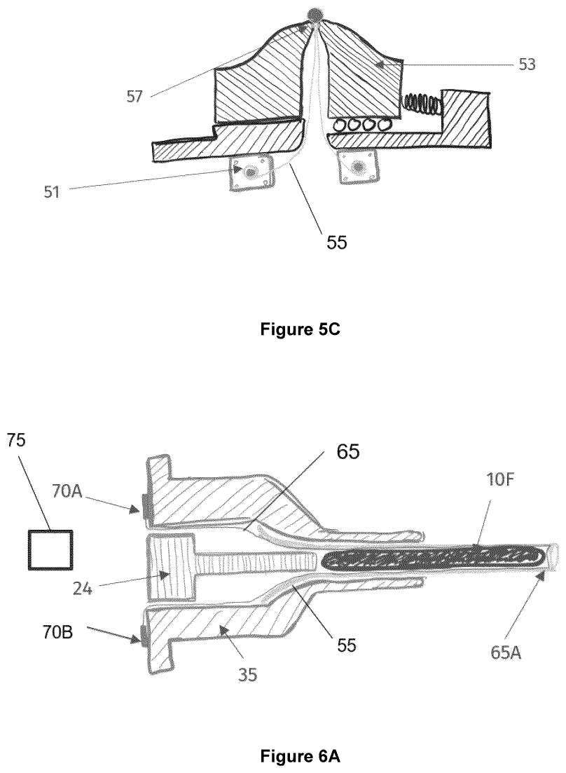

According to another aspect of the disclosure, as shown in FIG. 5A-B, an AIOL 10E is first placed into a compression device 50 that is designed to fold or compress the AIOL 10E prior to placement into the cartridge 40. The compressed AIOL, which may be wrapped in a thin film 55, is placed into the cartridge 40 so that the plunger 24 may deliver using any of the delivery systems or methods described herein.

In embodiments, the compressive device 50 allows a thin film 55 to surround the lens with two free ends running between a set of spring-loaded jaws 53, as shown in FIG. 5A. Pulling on the free end of the film 55 in an alternating fashion simultaneously rolls and compresses the lens allowing the stretch to be evenly distributed through the shell, as shown in FIG. 5B. The spring-loaded jaws 53 could have a resistive heating element 57 that can be used to heat seal the film into a tube around the lens once the desired compression is achieved. The free ends of the film 55 are then cut away leaving just the compressed lens in a thin film tube 59. Using the thin film as the tip of the inserter allows the tip to be much thinner than a conventional injection-molded inserter tip. FIG. 5C illustrates a cross-sectional schematic of another device 50 for packaging the lens in a thin film comprising spring loaded jaws 53, roller source(s) 51 of thin film 55, and an impulse heat sealer 57.

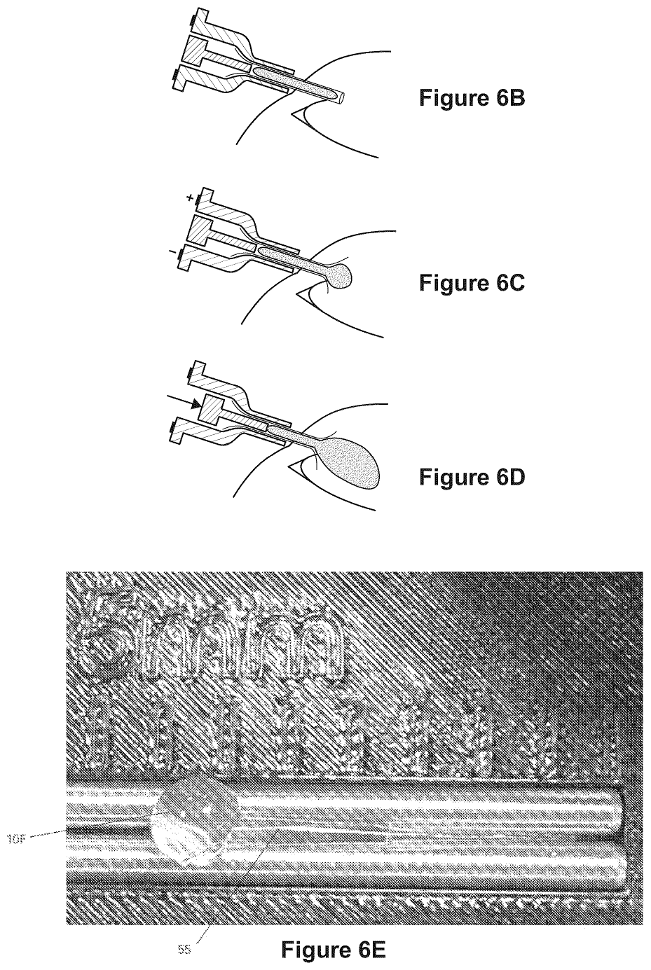

According to another aspect of the disclosure, FIG. 6A-E, an IOL 10F can be inserted into an eye using an expanding tip process. Referring to FIG. 6A, an injector body 20 or cartridge 35 has a lumen 25 opening at a distal end 26 thereof. An electrical contacts 70A-B are exposed on the injector body and configured to electrically couple a power source 75 associated with the delivery system with intraocular lens 10F and the wrapper assembly 73 disposed within the lumen 25. In embodiments, the intraocular lens delivery system is in combination an accommodating intraocular lens 10F comprising a flexible shell body filled with a filler liquid, the shell body at least partially compressed by a wrapper material and an electrically conductible element connectable to a power source. In embodiments, the assembly 73 comprises an accommodating intraocular lens at least partially compressed by a wrapper material 55 and an electrically conductible element 65 connectable to power source 75. In embodiments, at least a portion of wrapper material comprises a film. In embodiments, the electrically conductible element comprises a wire. In embodiments, coupling the electrically conductible element to the power source causes the wrapper to at least partially melt.

In embodiments, an IOL 10F is compressed into a thin film tube using the method described in herein with a resistive wire is embedded in the thin film and exposed to the film at the distal tip 65A. This assembly 73 is connected to an injection-molded polymer cartridge where the thin-film-coated-IOL is the only portion of the cartridge that is inserted into the eye. Once thin-film-coated IOL is inserted into the eye's capsular bag, the distal end of the IOL can be electronically released by passing a current through the resistive wire 65 and melting the tube of thin film 55 in a precise and controlled location and manner, allowing the lens to expand into the capsular bag. The IOL 10F can then be inserted using any of the delivery systems or methods described herein, without risk of overstretching or bursting the lens. Assembly 73 may be formed using methods previously described herein where only a portion of the lens is compressed within the thin film tube.

FIG. 6A schematically shows an inserter cartridge including a cartridge body 35, a plunger 24, wire 65, electrical contacts 70A-B, and an IOL 10F packaged in thin film 55. FIG. 6B schematically shows the IOL packaged in thin film being inserted through a small incision into the eye's capsular bag. FIG. 6C schematically shows the tip of the film 55 being cut when voltage is applied to the wire 65A inside the film. When the film is cut or melted, the distal end of the IOL is released into the eye's capsular bag. FIG. 6D schematically use of the plunger 24 to push the rest of the lens into the capsular bag. If the IOL 10F does not self-extrude into the eye's capsular bag, the plunger 24 may be s used to push the rest of the lens into the capsular bag. FIG. 6E is a photograph of a device for packaging the lens in a thin film. The device includes polyethylene film 55 run between two dowel pins to create a platform for rolling the IOL 10F into a small cylinder. In embodiments, element 65 may be implemented with a NiCr wire. In embodiments, the electrical contacts 70A-B may be located in the lumen 25 proximate the distal end 26 but still connectable to the power source 75.

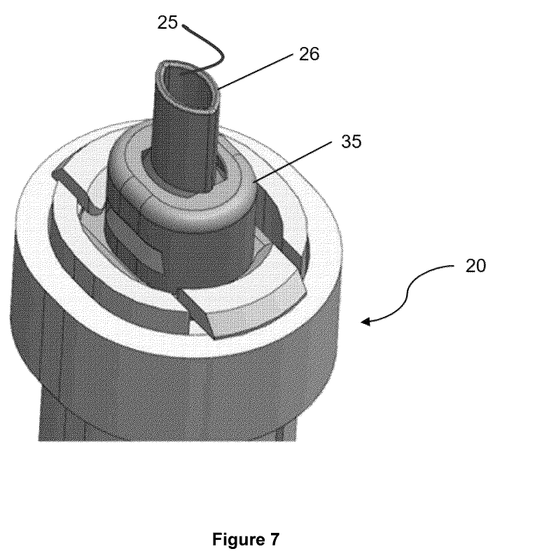

In embodiments, as illustrated in FIG. 7, the cross-sectional shape of the lumen 25 of cartridge 35 proximate the distal end 26 may be circular, oval, or even “football” shaped. The “football” shaped cartridge tip conforms generally to the shape of the incision in the eye, thereby maximizing volume within the tip and minimizing damage to the incision. FIG. 7 illustrates schematically the distal end 26 of cartridge 35 having a “football” shaped cross-sectional area. The inserter cartridge 35 performs multiple functions, including 1) providing a lumen to compress the IOL; 2) delivers the IOL through a small incision without damaging the incision site or the internal structures of the eye; 3) guides the IOL inside the eye so that it can be implanted directly into the capsular bag; and 5) guides the IOL during delivery such that it does not rotate during implantation.

Multiple features of the inserter cartridge 35 can impact performance of the lens delivery system including: 1) degree of taper of the cartridge lumen required to fold or compress the IOL; 2) dimensions of cartridge tip (length, diameter, radius, etc.); 3) shape of the cartridge tip to conform to shape of incision (circular, football or oval cross-sectional shaped); 4) any coating, blooming agent, or application of a lubricious surface treatment to minimize friction within the cartridge inner lumen; 5) surface texture or features on the surface of the inner lumen to minimize friction with the IOL; and 6) rigidity of the material from which the cartridge is made, e.g. some polymers can provide flexibility that assist in IOL delivery. In embodiments, other specific design feature may include a cartridge tip that splits or separates after it enters the eye to allow the IOL to open out of its compressed state once it is inside the eye.

According to another aspect of the disclosure, an IOL may be delivered by partial shell compression. The IOL is first placed into a compression device that is designed to fold or compress the IOL prior to placement into the cartridge 35. The compression device is then attached to the cartridge 35 so that the plunger 24 may deliver the lens by using any of the delivery systems or methods described herein.

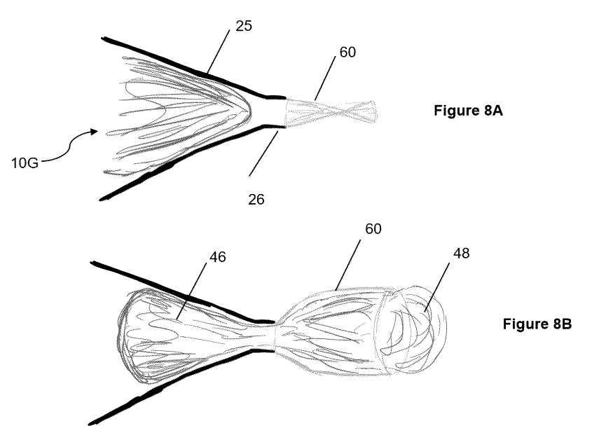

In accordance with another aspect of the disclosure, as illustrated in FIGS. 8A-B, a system for delivery of an intraocular lens 10G comprises a injector body having a lumen 25 opening exteriorly at a distal end 26 thereof, the lumen defined by an interior wall. A flexible tubular restraint 60 is coupled proximate the distal end 26 for directing expansion of an intraocular lens 10G emerging from the lumen in a controlled manner. In embodiments, the flexible tubular restraint 60 is attached to the interior wall of the lumen or at an exterior portion of the injector body. The flexible tubular restraint 60 is configured to prevent symmetric radial expansion of the intraocular lens. In embodiments, flexible tubular restraint comprises a foldable film. In embodiments, flexible tubular restraint 60 is any of wrapped, twisted or perforated in a predetermined pattern to bias expansion of the intraocular lens in a downward direction relative to an axis of the lumen 25 upon the intraocular lens emerging from the distal end 26. In embodiments, flexible tubular restraint is twisted into a predetermined expansion pattern. By expanding in a downward facing manner, the film and the expanding AIOL will not damage the corneal surface near the top of the capsular bag or deploy upside down. In embodiments, the IOL 10G may comprise an accommodating intraocular lens comprising a flexible shell body filled with a liquid.

FIGS. 8A-B illustrate a cross-sectional schematic of a system for reducing the length of the most narrow part of the distal end 26 and increasing the length of the larger-diameter section of lumen 25. In this approach, a thin film 60 that can be compressed or twisted down to a small diameter is attached to the tip of the inserter body 20. The short, distal tip 25 enters the incision site in the eye, e.g, <3.5 mm. The IOL 10G passes through the distal end 25, and, as the IOL 10G enters the film portion 60 inside of the eye, the IOL 10G expands to a larger diameter portion 48, allowing the surgeon to guide the lens into the capsular bag, but the portion of the inserter inside of the eye could be as little as 4-5 mm. The portion of the IOL that is most compressed will be shorter because the tip XX of the inserter body 20 inside of the eye will be flexible tubular restraint 60 that expands once inside the eye, allowing for the smallest diameter of the inserter, e.g. <3.5 mm, to be as short as possible because. Once inside the eye, the IOL will expand into the larger-diameter film portion of the inserter in a predetermined manner as defined by the configuration of the flexible tubular restraint 60. The flexible tubular restraint 60 may be any of wrapped, twisted or perforated in a predetermined pattern to bias the pattern in which the restraint 60 tears, and, ultimately the expansion pattern of the intraocular lens 10G in a downward direction, away from the cornea, relative to an axis of the lumen 25 upon the intraocular lens emerging from the distal end 26.

Although one or more of the delivery systems and methods have been described herein with reference to either an IOL or an AIOL, the reader will appreciate that both types of IOL may be utilized, as applicable, with the described delivery systems and methods.

As used herein, the term thread also encompasses strips or narrow widths of film or other materials capable of similar structural or functions behavior relative to an IOL or AIOL and any delivery system therefore.

The embodiments described above are intended to be merely exemplary; numerous variations and modifications will be apparent to those skilled in the art including, potentially, alternative geometries (e.g., to address different ocular conditions, eye shapes, eye sizes, etc.), alternative materials for the shell and/or the filler, etc. Such variations and modifications are intended to be within the scope of the present invention as defined by any of the appended claims. Any references to “invention” or “embodiments” are intended to be exemplary and should not be construed to refer to all possible embodiments unless the context otherwise requires. Accordingly, the described embodiments are to be considered in all respects only as illustrative and not restrictive. At various places in the present specification, values are disclosed in groups or in ranges. It is specifically intended that the description includes each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

For purposes of clarity and a concise description, features are described herein as part of the same or separate embodiments, however, it will be appreciated that scope of the concepts may include embodiments having combinations of all or some of the features described herein.

It will be apparent to those recently skilled in the art that modifications to the apparatus and process disclosed here in may occur, including substitution of various component values or nodes of connection, without parting from the true spirit and scope of the disclosure as defined by the claims set forth herein.

1. A system for delivery of an intraocular lens comprising, in combination:
an injector body having a lumen opening at a distal end thereof, the lumen having a minimum internal diameter; and
an accommodating intraocular lens comprising a flexible shell body filled with a filler liquid, the shell body is configured to at least partially assume an uncompressed maximum diameter substantially larger than the minimum internal diameter of the injector body lumen and further configured to at least partially assume a compressed diameter less than the minimum internal diameter of the injector body lumen.

2. The intraocular lens delivery system of claim 1 wherein the injector body further comprises a mechanism for moving the accommodating intraocular lens toward the distal end of the injector body lumen.

3. The intraocular lens delivery system of claim 2 wherein the accommodating intraocular lens is disposed within the injector body lumen distally of the mechanism for moving and proximally of the distal end of the injector body lumen.

4. The intraocular lens delivery system of claim 2 wherein the mechanism for moving the accommodating intraocular lens toward the distal end of the injector body lumen comprises a plunger slidably disposed within the injector body lumen.

5. The intraocular lens delivery system of claim 4 wherein the mechanism for moving the accommodating intraocular lens toward the distal end of the injector body lumen further comprises a liquid disposed within the injector body lumen between the plunger and the accommodating intraocular lens.

6. The intraocular lens delivery system of claim 5 wherein the injector body lumen comprises a wide diameter proximal section and a narrower diameter distal section in fluid communication therebetween, and wherein the plunger transfers distally directed force thereon through the fluid and to the accommodating intraocular lens causing compression of the shell body at a point of transition from the wide diameter proximal section to the narrower diameter distal section.

7. The intraocular lens delivery system of claim 2 wherein the mechanism for moving the accommodating intraocular lens toward the distal end of the injector body lumen further comprises a plurality of film strips disposed intermediate the minimum internal diameter of the injector body lumen and the at least partially compressed diameter the shell body.

8. The intraocular lens delivery system of claim 2 wherein the mechanism for moving the accommodating intraocular lens toward the distal end of the injector body lumen further comprises a mechanism for applying distally directed forces on the plurality of film strips.

9. The intraocular lens delivery system of claim 8 wherein the plurality of film strips transfer distally directed force thereon to the accommodating intraocular lens.

10.-27. (canceled)

28. A method for delivering an accommodating interocular lens comprising a flexible shell body filled with an incompressible liquid, the method comprising:
A) configuring a flexible tubular restraint attached to a distal end of a semi-rigid lumen into a predetermined expansion pattern, and
B) advancing an AIOL accommodating interocular lens in a compressed state out of a distal end of a semi-rigid lumen and into a flexible tubular restraint at a rate to prevent symmetric radial expansion of the intraocular lens upon emerging from the lumen.

29. The method of claim 28 wherein B) comprises:
B1) preventing expansion of the intraocular lens in an upward direction relative to an axis of the lumen upon the intraocular lens emerging from the distal end.

30. A method for delivering an intraocular lens comprising:
A) loading an intraocular lens into a lumen of an injector body, the lumen having an open distal end, and
B) advancing the intraocular lens distally through the lumen towards the open distal end without applying force to a proximal facing surface of the intraocular lens as positioned in the lumen.
